(19) United States
(12) Patent Application Publication
(10) Pub. No.: US 2026/0049286 A1
(43) Pub. Date: Feb. 19, 2026

(54) FASL-MODIFIED PLG SCAFFOLDS ENHANCES DIFFERENTIATION OF STEM CELL DERIVED BETA CELLS

(71) Applicants: THE CURATORS OF THE UNIVERSITY OF MISSOURI, Columbia, MO (US); THE REGENTS OF THE UNIVERSITY OF MICHIGAN, Ann Arbor, MI (US)

(72) Inventors: Haval SHIRWAN, Columbia, MO (US); Esma YOLCU, Columbia, MO (US); Lonnie SHEA, Ann Arbor, MI (US)

(21) Appl. No.: 19/101,972

(22) PCT Filed: Aug. 8, 2023
(86) PCT No.: PCT/US2023/029768

§ 371 Date: Feb. 7, 2025

Related U.S. Application Data
(60) Provisional application No. 63/370,724, filed on Aug. 8, 2022.

(51) Int. Cl. C12N 5/071; A61K 35/39; A61K 47/69

(52) U.S. Cl. CPC C12N 5/0676; A61K 35/39; A61K 47/6937; C12N 2533/40; C12N 2533/50

(57) ABSTRACT

The present disclosure is generally directed to the use of biomaterial scaffolds engineered with SA-FasL for the transplantation of stem cell derived β-cells as a treatment for Type I diabetes. Early engraftment post-transplantation and subsequent maturation of these β-cells may be limited by the initial inflammatory response, which impacts the ability to sustain normoglycemia at long times. The survival and development of immature hPSC-derived β-cells transplanted on poly(lactide-co-glycolide) (PLG) microporous scaffolds into the peritoneal fat, a site being considered for clinical translation, was investigated. The scaffolds were modified with biotin for binding of a streptavidin-FasL (SAFasL) chimeric protein to modulate the local inflammatory microenvironment. The presence of FasL impacted infiltration of monocytes and neutrophils and altered their phenotypic response. Conditioned media generated from scaffolds explanted at day 4 did not impact hPSC-derived β-cell survival and maturation in vitro, which was not observed with unmodified scaffolds. Following transplantation, β-cell viability and differentiation were improved with SA-FasL modification. A sustained increase in insulin positive cell ratio was observed with SA-FasL modified relative to unmodified scaffolds. These results demonstrate that SA-FasL-modified scaffolds can mitigate initial inflammatory response and enhance β-cell engraftment and differentiation.

### CROSS REFERENCE TO RELATED APPLICATIONS

This application claims the benefit of U.S. Provisional Application No. 63/370,724, filed Aug. 8, 2022, the contents of which are incorporated by reference herein in their entirety.

### STATEMENT REGARDING FEDERALLY SPONSORED RESEARCH OR DEVELOPMENT

This invention was made with government support under AI147677 awarded by the National Institutes of Health. The government has certain rights in the invention.

### FIELD OF INVENTION

The present disclosure relates to treatment of Type I diabetes. Particularly, this disclosure is directed to using poly(lactide-co-glycolide) (PLG) scaffolds engineered with streptavidin (SA)-FasL for the transplantation of stem cell derived β-cells as a treatment for Type I diabetes.

### BACKGROUND OF INVENTION

Type I diabetes (TID), which affects an estimated 1.9 million Americans, is an autoimmune disease involving a progressive destruction of pancreatic islet clusters, leading to loss of insulin production and secretion. The standard treatments for TID, involves a life-long administration of exogenous insulin, either by daily insulin injections or an insulin pump, which are often associated with hypoglycemic episodes that contribute to complications. Several secondary metabolic disorders can arise from this disease, such as retinopathy, neuropathy, nephropathy, stroke and heart failure. Allogeneic islet transplantation has led to the transient reversal of diabetes and improved glucose control, which contribute to a decline in secondary complications. Although allogeneic islet transplantation holds great therapeutic potential for TID, a major limitation lies with the shortage of donor islets.

The shortage of donor islets has led to the development of human pluripotent stem cells (hPSCs) as an unlimited source to deliver functional B-cells. The feasibility of differentiating hPSCs to immature β-cells in vitro and further maturation of the immature β-cells to insulin-producing β-cells in vivo has been reported. These studies are typically performed with transplantation into the kidney capsule, a site that is not considered translational. Clinical islet transplantation, and recently the delivery of stem cell derived beta cells, are performed by delivery through the portal vein into the liver. This site has supported cell function and reduction of exogenous insulin, yet has limitations that include cell loss or dysfunction due to the instant blood mediated inflammatory reaction (IBMIR) and elevated levels of toxins including metabolites and pharmaceuticals employed to prevent rejection. Extrahepatic transplantation, such as cell delivery into the peritoneal fat or subcutaneous space, is being investigated to address these limitations.

Enhancing the efficacy of transplantation for islets and stem cell derived β-cells has been investigated using biomaterials, particularly at extrahepatic sites. Encapsulation approaches can prevent direct contact between grafts and immune cells, protecting islets from the host immune response. Alternatively, microporous scaffolds have been used to integrate the transplanted cells with the host tissue, and provide a three-dimensional (3D) support for cell adhesion and differentiation, which has enhanced β-cell engraftment. Nevertheless, inflammatory responses at these sites are recognized as hindering engraftment of islets post transplantation, and stem cell derived β-cells are particularly sensitive to inflammatory signals. Soluble mediators such as inflammatory cytokines secreted by recruited innate cells can harm the transplanted cells. Factors such as FasL, IL-10, IL-33, TGF-β1 and dexamethasone have been delivered to improve islet survival and function by modulating the immunological environment around scaffolds. Materials modified with streptavidin-Fas Ligand (SA-FasL) have supported engraftment of transplanted islets that restored normoglycemia for 200 days without the need for sustained immune suppression. FasL has been typically associated with modulating T cell responses, yet this modification of the biomaterial may alter early inflammatory responses by innate immune cells.

### SUMMARY OF INVENTION

This disclosure describes a composition comprising a FasL-loaded poly(lactide-co-glycolide) (PLG) scaffold and immature β-cells.

The present disclosure is further directed to a method of making a composition comprising a FasL-loaded poly(lactide-co-glycolide) (PLG) scaffold and immature β-cells, wherein the method comprises:
a) loading poly(lactide-co-glycolide) (PLG) with biotin;
b) making a biotin-loaded PLG scaffold from the biotin-loaded PLG;
c) conjugating streptavidin-FasL to the biotin on the biotin-loaded PLG scaffold to form a FasL-loaded PLG scaffold; and
d) seeding the FasL-loaded PLG scaffold with immature β-cells.

The present disclosure is also directed to a method of treating a subject with type I diabetes, wherein the method comprises implanting a composition comprising a FasL-loaded PLG scaffold and immature β-cells into the subject.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1A-D depict how insulin-producing β cells were differentiated through a 6-stage protocol from hPSCs. Differentiated cells'quality was checked at multiple stages for protein expressions using flow cytometry analysis. FIG. 1A depicts how progenitors were expressing 98% OCT3/4+, 67% of PDX1+/NKX6.1+, 45% CHGA+/NKX6.1+, and 46% C-pep+/NKX6.1+ by the end of stage 0, 4, 5, and 6 respectively. FIG. 1B depicts how gene expressions by PCR analysis indicate functional hPSC-derived β cells produced at the end of stage 6, n=7. FIG. 1C depicts how gene expressions by glucose-stimulated insulin secretion indicate functional hPSC-derived β cells produced at the end of stage 6, n=7. FIG. 1D depicts how six million stage 6 cells were seeded into PLG scaffolds right before the transplantations with over five million of the seeded β cells remaining in the PLG scaffold.

FIGS. 2A-C depict in vivo immune infiltration into seeded PLG scaffolds. The PLG scaffolds seeded by immature β cell were transplanted to the left (FasL) and right (SA) epididymal fat pads of NSG mice. The scaffolds were harvested on day 4, 7, and 14, and the infiltrated cells were extracted and characterized. *P <0.05, **P<0.01, ****P<0.0001, Student's t test. FIG. 2A depicts a representative flow cytometry analysis of scaffolds harvested on day 4 for characterization of F4/80+ macrophages, Ly6C+ monocytes, and Ly6G+ neutrophils. FIG. 2B depicts the population of infiltrated immune cells from SA-FasL-engineered or SA-scaffolds on day 4, 7, and 14, n=5. FIG. 2C depicts RT-PCR analysis of the indicated transcripts in the infiltrated macrophages, n=8.

FIGS. 3A-D depict an illustration of conditioned media timeline and viability of the hPSC-derived immature β cells. SA-FasL-scaffolds and SA-scaffolds loaded with hPSC-derived β cells were extracted four days after transplantation and incubated in stage 6 media for conditioning. Fresh stage-6 β cells were co-cultured with the conditioned media for 2 days and counted by the DNA quantification method. **P<0.01, ****P<0.0001, Student's t test. FIG. 3A depicts an illustration of the timeline for the conditioned media culture. FIG. 3B depicts the total cell number from each conditioned co-culture by PicoGreen dsDNA Assay, n=8 for control, n=7 for SA-FasL, n=3 for SA. FIG. 3C-3D depict apoptotic and dead cells measured by propidium iodide and Annexin V staining and flow cytometry analysis, n=4. For comparison, stage-6 β cells co-cultured with non-conditioned fresh stage 6 media was added as the control. FIG. 3E depicts how caspase-8 expression level was quantified in the β cells treated by different conditions, n=8.

FIGS. 4A-C depict β-cell lineage biomarkers of the hPSC-derived immature β cells from conditioned media. As described previously, conditioned media was prepared by the extracted scaffolds. *P<0.05, **P<0.01, Student's t test. FIGS. 4A-4B depict how stage-6 β cells were co-cultured with the conditioned media for 2 days and maturation markers, NKX6.1 and INS, were analyzed by flow cytometry, n=4. Control was stage-6 β cells co-cultured with fresh, non-conditioned stage 6 media. FIG. 4C depicts how PCR analysis was used to measure the key β-cell lineage gene expression across the conditions, n=4.

FIG. 5 depicts in vivo human cell viability on the PLG scaffolds. Immunohistochemistry sections of harvested SA-FasL-engineered scaffolds and harvested SA-engineered scaffolds from day 4, 7, and 14 were stained with a human mitochondrial protein to distinguish the human from mouse cells and then quantified. FIG. 5 depicts the human cell numbers quantified using MATLAB software, n=3. *P<0.05, Student's t test.

FIGS. 6A-B depict image quantification of monohormonal insulin producing cells and polyhormonal insulin/glucagon producing cells for the extracted SA-FasL- and SA-scaffolds on day 4, 7, and 14 post-transplantation. *P<0.05, ***P<0.001, ****P<0.0001, Student's t test. Immunohistochemistry sections of SA-FasL coated scaffolds and SA-scaffolds were stained for insulin, glucagon, and counterstained with DAPI. FIGS. 6A-6B depict histograms of scaffolds at varied time points, n=4.

FIG. 7 depicts transcript levels of the indicated genes in β cells seeded in SA-FasL- and SA-scaffolds as assessed by RT-PCR on day 14 post-transplantation, n=4. *P<0.05, ***P<0.001, Student's t test.

### DETAILED DESCRIPTION OF INVENTION

Stem cell derived β-cells have demonstrated the potential to control blood glucose levels and represent a promising treatment for Type I diabetes (TID). Early engraftment post-transplantation and subsequent maturation of these β-cells are hypothesized to be limited by the initial inflammatory response, which impacts the ability to sustain normoglycemia for long periods. The survival and development of immature hPSC-derived β-cells transplanted on poly(lactide-co-glycolide) (PLG) microporous scaffolds into the peritoneal fat, a site being considered for clinical translation, were investigated. The scaffolds were modified with biotin for binding of a streptavidin-FasL (SA-FasL) chimeric protein to modulate the local inflammatory microenvironment. The presence of FasL impacted infiltration of monocytes and neutrophils and altered their phenotypic response. Conditioned media generated from scaffolds explanted at day 4 post-transplant did not impact hPSC-derived β cell survival and maturation in vitro, whereas conditioned media from unmodified scaffolds did. Following transplantation, β-cell viability and differentiation were improved with SA-FasL modification. A sustained increase in insulin positive cell ratio was observed with SA-FasL modified scaffolds relative to unmodified scaffolds. These results highlight that the initial inflammatory response can significantly impact β-cell engraftment and development, and modulation of this response may be a consideration for supporting long-term function at an extrahepatic site.

Additionally, the SA-FasL modification of microporous PLG scaffolds was investigated for its ability to enhance short-term (i.e., two weeks) engraftment and maintain differentiation of the transplanted stem cell derived β-cells. The initial period post-transplantation is important and can influence the fate of transplanted stem cell derived β-cells. The cell laden scaffolds were transplanted into an extrahepatic site, the epididymal fat pad. The epididymal fat pad in mice, which has many features similar to human white adipose tissue found in the omentum, has been recognized as a potential transplant site for islet transplantation in a recent clinical study because of its accessible location, immune environment, and capacity to accommodate high tissue volume. The microporous PLG scaffolds were biotinylated for subsequent immobilization of SA-FasL. Both the transplanted immature β-cells and the infiltrated mouse immune cells were analyzed at multiple time points post-transplantation. These studies highlight the impact of the local immune environment on the engraftment and differentiation of transplanted hPSC-derived immature β-cells.

This disclosure describes a composition comprising a FasL-loaded poly(lactide-co-glycolide) (PLG) scaffold and immature β-cells.

The immature β-cells can be human pluripotent stem cell (hPSC)-derived cells. The immature β-cells can be human induced pluripotent stem cell (iPSC)-derived cells or any cells producing insulin. The immature β-cells can be derived according to the methods described herein.

The immature β-cells can comprise about 0.1 million to about 10 million immature β-cells per scaffold; from about 0.5 million to about 10 million immature β-cells per scaffold; from about 1 million to about 10 million immature β-cells per scaffold; from about 0.5 million to about 8 million immature β-cells per scaffold; from about 0.5 million to about 5 million immature β-cells per scaffold. Further, the immature β-cells comprise at least six million immature-cells per scaffold.

For a human patient, the number of immature β-cells administered to the patient can comprise about 400 million to about 800 million immature β-cells.

Accordingly, the number of scaffolds administered to the human patient would be calculated to provide the appropriate number of immature β-cells. For example, the number of scaffolds administered to a human patient could be from about 20 scaffolds to about 2500 scaffolds, from about 20 scaffolds to about 2000 scaffolds, from about 20 scaffolds to about 1800 scaffolds, from about 20 scaffolds to about 1500 scaffolds, from about 20 scaffolds to about 1200 scaffolds, from about 20 scaffolds to about 1000 scaffolds, from about 20 scaffolds to about 800 scaffolds, from about 20 scaffolds to about 500 scaffolds, or from about 20 scaffolds to about 200 scaffolds.

The FasL-loaded PLG scaffold can be from about 1 mm in diameter to about 50 mm in diameter. In certain embodiments, the FasL-loaded PLG scaffold can be about 5 mm in diameter or between about 10 mm to about 35 mm in diameter. The FasL-loaded PLG scaffold can be from about 0.5 mm in height to about 5 mm in height. In certain embodiments, the FasL-loaded PLG scaffold is about 2 mm in height. The FasL-loaded PLG scaffold can have a pore size of between about 100 microns and about 600 microns. In certain embodiments, the FasL-loaded PLG scaffold has a pore size of between about 250 microns and about 425 microns.

The FasL-loaded PLG scaffold can comprise at least 0.1 μg of FasL. In certain embodiments, the FasL-loaded PLG scaffold comprises at least 1 μg of FasL. In certain embodiments, the FasL-loaded PLG scaffold comprises about 1 μg to about 50μg of FasL. The FasL loaded onto the PLG scaffold can be SA-FasL or another form of FasL known in the art.

The composition can further comprise more than one FasL-loaded PLG scaffold. In certain embodiments, the composition can further comprise at least two FasL-loaded PLG scaffolds or at least 10 FasL-loaded PLG scaffolds.

The present disclosure is further directed to a method of making a composition comprising a FasL-loaded poly(lactide-co-glycolide) (PLG) scaffold and immature β-cells, wherein the method comprises:
a) loading poly(lactide-co-glycolide) (PLG) with biotin;
b) making a biotin-loaded PLG scaffold from the biotin-loaded PLG;
c) conjugating streptavidin-FasL to the biotin on the biotin-loaded PLG scaffold to form a FasL-loaded PLG scaffold; and
d) seeding the FasL-loaded PLG scaffold with immature β-cells.

Loading the PLG with biotin can comprise activating the carboxyl end group of PLG with 1-ethyl-3-(3-dimethylaminopropyl)carbodiimide hydrochloride (EDC) followed by N-hydroxysuccinimide (NHS) and then adding amine-PEG2-biotin.

Alternatively, the PLG scaffold can be loaded with FasL by any conjugation method, chemical modification, or other means known in the art.

Making the biotin-loaded scaffold from the biotin-loaded PLG can comprise:
a) combining the biotin-loaded PLG with unmodified PLG at a mass ratio of 3:1 and forming biotin-loaded PLG particles,
b) mixing the biotin-loaded PLG particles with NaCl at a mass ratio of 1:30 and pressing the mixture into a 5 mm KBr die to form the biotin-loaded scaffold; and
c) disinfecting the biotin-loaded scaffold by soaking it in 70% ethanol.

Conjugating streptavidin-FasL to the biotin on the biotin-loaded PLG scaffold can comprise adding about 1 μg to about 50 μg streptavidin-FasL diluted in PBS to the biotin-loaded PLG scaffold and incubating it at 20° C. for 30 minutes while rotating and shaking the plate every 10 minutes.

Seeding the FasL-loaded PLG scaffold with immature-cells can comprise adding immature β-cells at a density of from about 1 million to about 10 million cells per 30 μL of media and seeding from about 0.5 million to about 5 million cells per side of the FasL-loaded PLG scaffold.

Further, seeding the FasL-loaded PLG scaffold with immature β-cells can comprise adding immature β-cells at a density of about 6 million cells per 30 μL of media and seeding about 2.5 million cells per side of the FasL-loaded PLG scaffold.

In certain embodiments, the FasL-loaded PLG scaffolds with immature β-cells can comprise a total of about 400 million to about 800 million immature β-cells. The immature-cells can be hPSC-derived cells. The immature β-cells can be human iPSC-derived cells or cells producing insulin.

The present disclosure is also directed to a method of treating a subject with type I diabetes, wherein the method comprises implanting a composition comprising a FasL-loaded PLG scaffold and immature β-cells into the subject.

The scaffold can be transplanted into the subject's peritoneal fat. In certain embodiments, the scaffold is transplanted into white adipose tissue of the subject's omentum or epididymal fat pad. The subject can be a rodent, non-human primate, or human. In certain embodiments, the subject is a human.

The immature β-cells can be hPSC-derived cells. The immature β-cells can be human iPSC-derived cells or any cells producing insulin. In certain embodiments, the immature β-cells are derived from the subject via iPSCs. The immature β-cells can be derived according to the methods described herein. The immature β-cells can comprise about 400 million to about 800 million immature β-cells. The immature-cells can comprise about 1 million to about 10 million immature β-cells. In certain embodiments, the immature β-cells comprise at least six million immature β-cells.

The FasL-loaded PLG scaffold can be from about 1 mm in diameter to about 50 mm in diameter, from about 1 mm to about 45 mm, from about 1 mm to about 40 mm, from about 1 mm to about 35 mm, from about 5 mm in diameter to about 50 mm in diameter, from about 5 mm to about 45 mm, from about 5 mm to about 40 mm, from about 5 mm to about 35 mm, from about 10 mm in diameter to about 50 mm in diameter, from about 10 mm to about 45 mm, from about 10 mm to about 40 mm, or from about 10 mm to about 35 mm in diameter. In certain embodiments, the FasL-loaded PLG scaffold can be about 5 mm in diameter or between about 10 mm to about 35 mm in diameter. The FasL-loaded PLG scaffold can be from about 0.5 mm in height to about 5 mm in height. In certain embodiments, the FasL-loaded PLG scaffold is about 2 mm in height. The FasL-loaded PLG scaffold can have a pore size of between about 100 microns and about 600 microns. In certain embodiments, the FasL-loaded PLG scaffold has a pore size of between about 250 microns and about 425 microns.

The FasL-loaded PLG scaffold can comprise at least 0.1 μg of FasL. In certain embodiments, the FasL-loaded PLG scaffold comprises at least 1 μg of FasL. In certain embodiments, the FasL-loaded PLG scaffold comprises about 1 μg to about 50μg of FasL. The FasL loaded onto the PLG scaffold can be SA-FasL or any other form of FasL known in the art.

The composition can further comprise more than one FasL-loaded PLG scaffold. In certain embodiments, the composition can further comprise at least two FasL-loaded PLG scaffolds or at least 10 FasL-loaded PLG scaffolds.

As used in this application, including the appended claims, the singular forms “a,” “an,” and “the” include plural references unless the content clearly dictates otherwise, and are used interchangeably with “at least one” and “one or more.”

The invention will be further described in the following examples, which do not limit the scope of the invention described in the claims.

### EXAMPLES

It is to be understood that while the invention has been described in conjunction with the detailed description thereof, the preceding description is intended to illustrate and not limit the scope of the invention, which is defined by the scope of the appended claims. Other aspects, advantages, and modifications are within the scope of the following claims.

### Example 1: Cell Production and Scaffold Seeding

A 6-stage differentiation protocol was employed to culture pluripotent stem cells into immature β-cells (Hogrebe, N. J., et al., Nat. Biotechnol. 38, 460-470 (2020); Millman, J. R. et al., Nat. Commun. 2016 71 7, 1-9 (2016); Bealer, et al., Biomater. Sci. 2023 11, 3645-3655).

Briefly, undifferentiated cells were single-cell dispersed and seeded at 5×10⁶ cells/mL in 6-well cell culture treated plates coated with Matrigel (Coming). Cells were cultured for 72 h in mTeSR1 and then cultured in the differentiation media for 6 stages. During each stage, cells received daily media changes with stage-specific basal media supplemented with growth factors and small molecules to induce differentiation. The stages of this protocol are defined as definitive endoderm (stage 1), primitive gut tube (stage 2), pancreatic progenitor (stages 3 and 4), endocrine progenitor (stage 5), and immature stem cell derived-cell (stage 6).

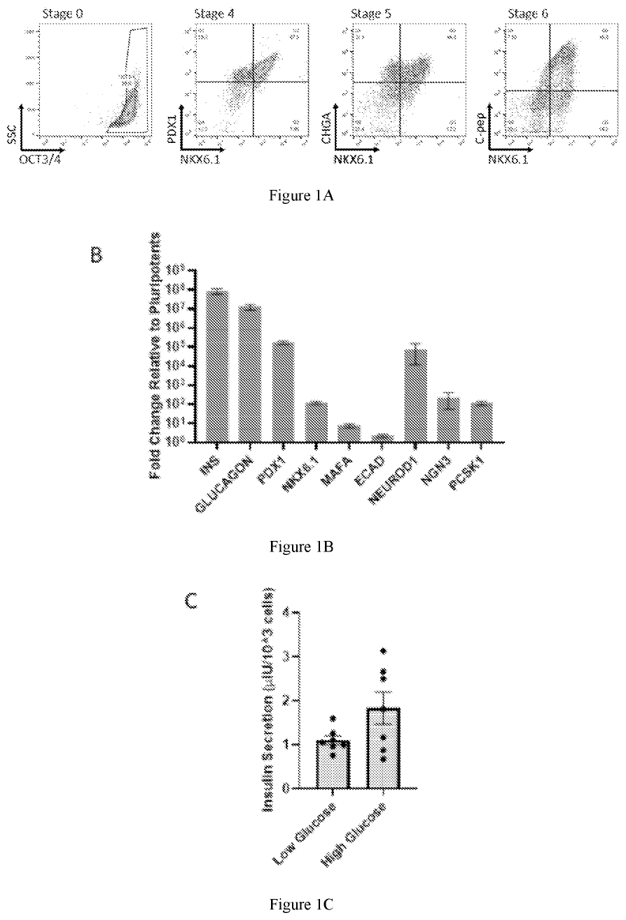

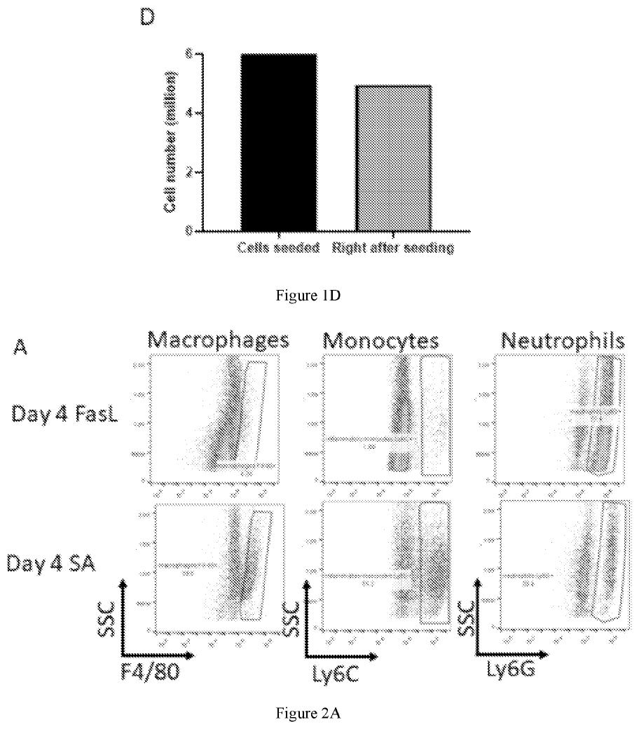

hPSC-derived cells were analyzed at multiple time points during the differentiation protocol for gene expression and indicated 98% OCT3/4⁺, 67% of PDX1⁺/NKX6.1⁺, 45% CHGA⁺/NKX6.1⁺, and 46% C-pep⁺/NKX6.1⁺ populations at the end of stage 0, 4, 5, and 6 respectively (FIG. 1A). At day 10 of stage 6, the gene expression of the differentiation was further analyzed by PCR with INS increased 10⁷ fold compared to pluripotent cells (FIG. 1B). Insulin secretion in the presence of both low and high glucose levels (2.0 mM and 20 mM glucose solutions) increased from averaging 1.11 μU/10³ cells to 1.83 μU/10³ cells when stimulated by different glucose levels (FIG. 1C). Cell number quantified after seeding determined the seeding efficiency exceeded 80% (FIG. 1D).

### Example 2: SA-FasL-Engineered Scaffolds Modulate Innate Immune Responses

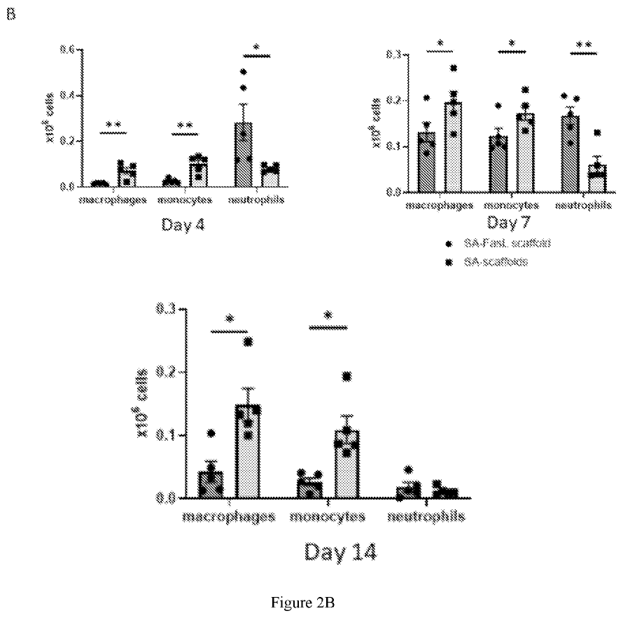

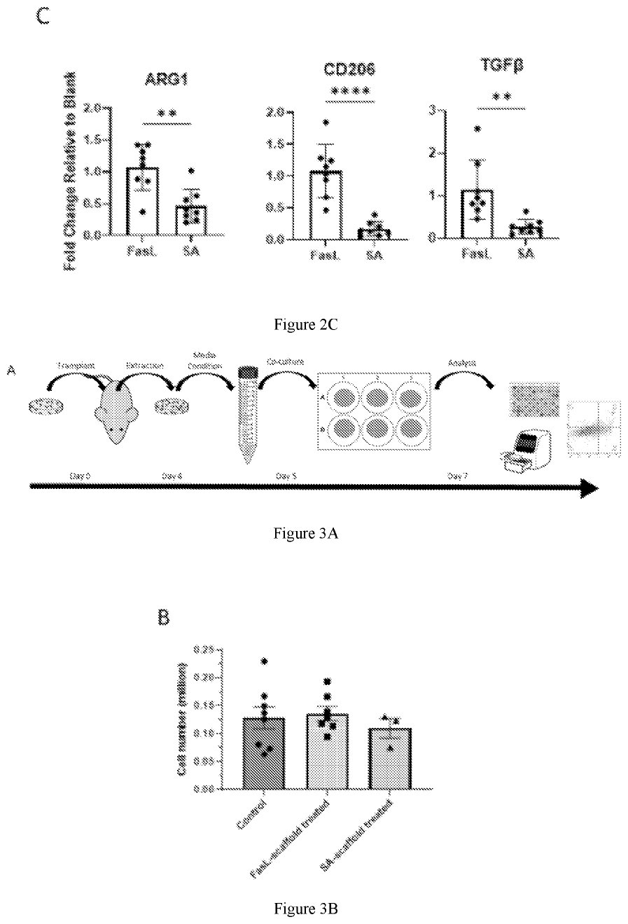

Next modulation of the local microenvironment by SA-FasL was investigated, as most innate cells upregulate Fas upon activation and can serve as target for FasL-mediated apoptosis. Cell-scaffold constructs were created by immobilizing SA-FasL to biotinylated scaffolds, loaded with Stage 6 β-cells, and transplanted into epididymal fat pad of NSG mice deficient for adaptive immunity. Significantly decreased numbers of macrophages and monocytes were identified in the SA-FasL-engineered scaffolds relative to the SA-scaffolds (FIG. 2A, B). Macrophage and monocyte abundance in the scaffold was maximal at day 7 post transplantation. The infiltrated macrophages at day 4 post-transplant were analyzed for expression of Arginase 1 (ARG1), CD206, and transforming growth factor β (TGF-β), which are associated with an anti-inflammatory phenotype, and the expression of these genes was significantly greater in SA-FasL-engineered scaffolds, suggesting macrophage polarization towards an anti-inflammatory (M2) phenotype (FIG. 2C), For neutrophils, the abundance of neutrophils was increased at days 4 and 7 post-transplantation for the SA-FasL scaffolds relative to SA-scaffolds, with the neutrophil abundance similar for the two scaffold conditions at day 14.

### Example 3: In Vitro Assessment of Inflammation on B-Cells

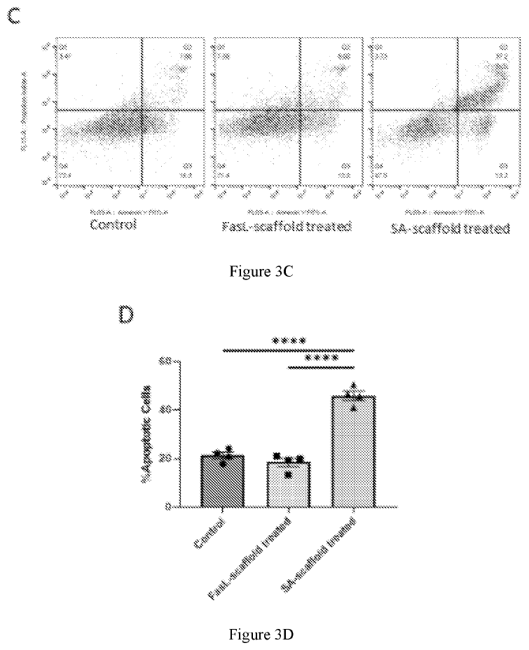

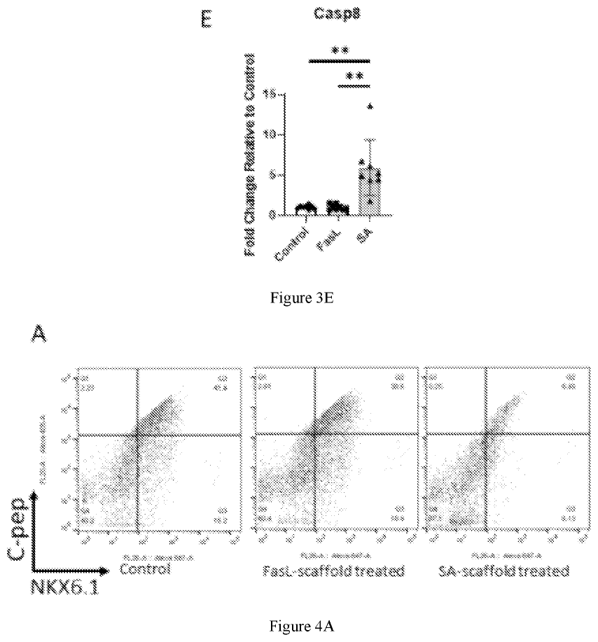

The impact of the immune cell response on the stem cell derived β-cells was analyzed in vitro through the generation of media conditioned by cells from extracted scaffolds. Scaffolds engineered with SA as a control protein or SA-FasL were loaded with stem cell derived β-cells and transplanted into the epididymal fat pad, with each mouse receiving one seeded SA-scaffold and one seeded SA-FasL-scaffold in different sites. The scaffolds were explanted, placed into culture for conditioning of the media (FIG. 3A), and this conditioned media was subsequently applied to Stage 6 β-cells for analysis of cell viability and apoptosis. The control condition is where the fresh, non-conditioned culture media was used. Total cell number was consistent across scaffold conditions (FIG. 3B), with a greater percentage of viable cells present in SA-FasL-scaffold conditioned media compared to SA-scaffold (FIG. 3C). The percentage of apoptotic cells (Annexin V+) was significantly greater in the SA-scaffold condition compared to both control and SA-FasL-scaffolds (FIG. 3C, D). Caspase-8 expression was also increased in the SA-scaffold condition (FIG. 3E), indicating an increased apoptosis through the extrinsic pathway, supporting a correlation between increased inflammation in the microenvironment and cell death.

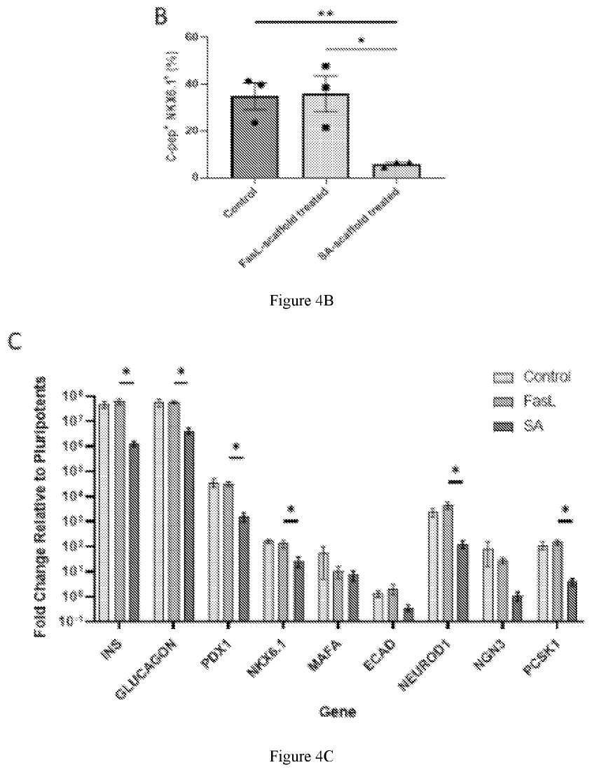

The impact of the infiltrated immune cells on the stem cell derived β-cell expression of differentiation associated genes was next investigated. Stem cell derived β-cells co-cultured with the SA-FasL-scaffolds conditioned media had a greater percentage of cells that were C-pep+/NKX6.1+ relative to those treated with SA-scaffolds conditioned media (FIG. 4A, B), with the percentage of C-pep+/NKX6.1+ cells similar between the SA-FasL-scaffolds conditioned media and the normal culture media. PCR was employed to more broadly assess the expression of differentiation and maturation markers. Cells cultured with the SA-FasL-scaffolds conditioned media had higher expression for INS, GLUCAGON, PDX1, NKX6.1, NEUROD1, and PCSK1 compared to cultures with the SA-scaffolds conditioned media (FIG. 4C). Overall, the improved expression of differentiation markers and decreased levels of apoptosis indicated that SA-FasL-scaffolds may better support the engraftment of stem cell derived β-cells post-transplantation.

### Example 4: In Vivo Assessment of Transplanted β-Cells

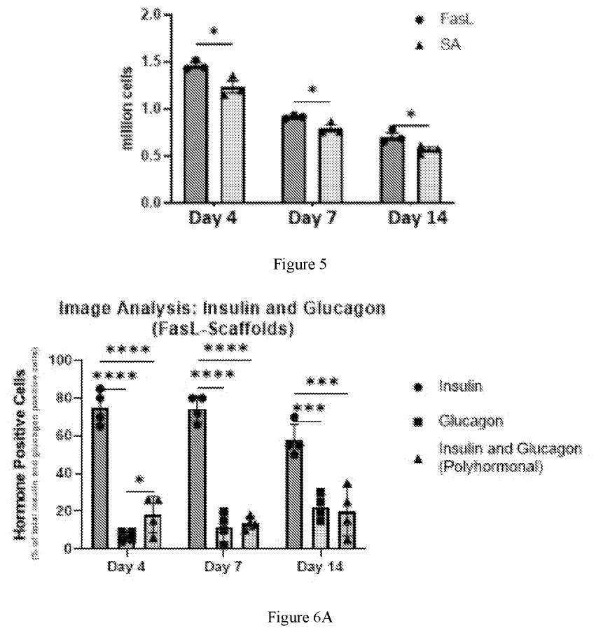

SA-FasL-engineered scaffolds and their immune modulation were subsequently investigated for an impact on cell engraftment and post-transplantation differentiation towards mature β-cells. SA-FasL-engineered scaffolds retrieved at days 4, 7, and 14 post-transplantation demonstrated substantial numbers of cells that were positive for DAPI and co-stained for human nuclei (HuNu), indicating a transplanted cell. These cells were present throughout the polymer scaffolds, and human cells were not identified outside of the scaffold. The SA-FasL-scaffolds retained a greater number of the transplanted cells relative to the SA-scaffolds (FIG. 5). The number of the transplanted cells was greater by approximately 20% on the SA-FasL-scaffolds relative to the SA-scaffolds, and the cell numbers decreased with time of transplantation, averaging 1.46 million on day 4 to 0.71 million on day 14 within a SA-FasL-scaffold or from 1.24 million to 0.57 million within an SA-scaffold.

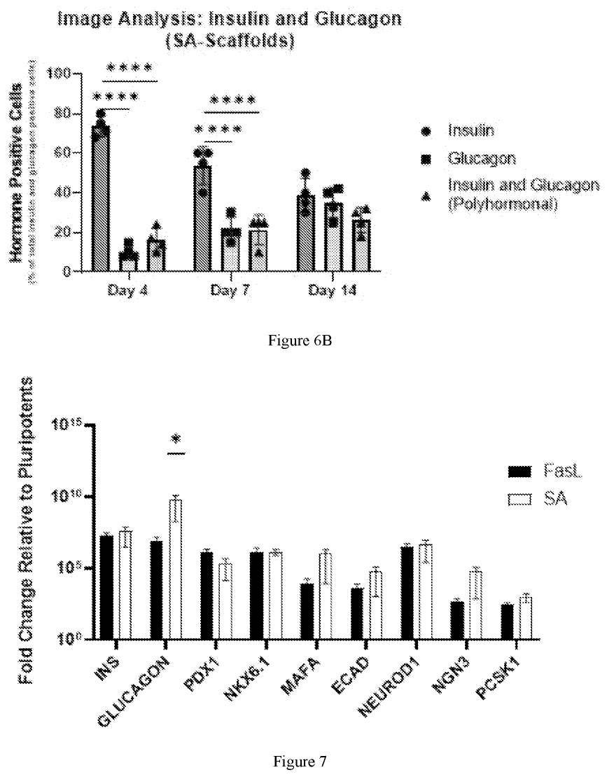

The numbers of hormone (i.e., insulin, glucagon) positive cells were subsequently analyzed for the SA- and SA-FasL-scaffolds (FIG. 6A-B). The insulin positive cells outnumbered the glucagon positive and the dual insulin and glucagon positive cells at all three timepoints for the SA-FasL-scaffolds (FIG. 6A-B). The monohormonal insulin positive cell ratio on the SA-FasL-scaffolds decreased from approximately 75% of total hormonal positive cells on day 4 or day 7 to 57.5% on day 14. By day 14, monohormonal insulin positive cells averaged 57.5% and 38.8% of the hormone positive cells on SA-FasL- and SA-scaffolds respectively (p=0.0216) with total insulin positive cells (including both monohormonal and polyhormonal) averaged 77.5% on SA-FasL condition and 65% on SA condition. Glucagon positive cells averaged 42.5% and 61.25% of hormone positive cells between SA-FasL- and SA-scaffolds, respectively. The percentage of hormone positive cells was significantly different between SA-FasL- and SA-scaffolds only for the glucagon positive cells (p=0.022). The differentiation state of the stem cell derived β-cells was analyzed on day 14 post transplantation. Gene expression was measured on day 14 post transplantation, at which time point significant differences were observed in the expression of glucagon between the SA-FasL- and SA-scaffolds, which is consistent with the increased percentage of glucagon positive cells for the SA-scaffolds relative to the SA-FasL-scaffolds (FIG. 7). No significant differences between the SA-FasL-and SA-scaffolds were observed for expression of the other eight genes analyzed.

### Example 5: Conclusions

This report investigated the initial survival and engraftment of transplanted immature β-cells, with and without modulation of the innate immune response following transplantation. Microporous polymer scaffolds were engineered to positionally display SA-FasL on their surface, which has been previously been employed to support long term engraftment and function of transplanted allogeneic islets without sustained immune suppression in rodents and recently nonhuman primate models. The SA-FasL-based immunomodulatory regimens have been reported as an effective means of culling alloreactive T effector cells while increasing the anti-inflammatory activity of CD4+CD25+FoxP3+ T regulatory cells. Herein, this platform was used to transplant human pluripotent stem cell (hPSC) derived β-cells into the epididymal fat pad, a clinically relevant site and equivalent of the human greater omentum. Multiple protocols have been established for production of pancreatic progenitors and β-cells in vitro, yet these β-cells are not fully mature following in vitro culture, and the local transplant environment can influence their fate. β-cells are more susceptible to inflammatory insults than islets. Inflammation has been associated with detrimental effects to hPSC derived-cells following transplantation, including reduced survival and alteration of the differentiation capacity.

In this study, it was demonstrated that SA-FasL engineered scaffolds modulated the innate immune environment, and that the factors secreted were not detrimental to the survival and function of hPSC-derived β-cells, in contrast to what was observed with control SA-engineered scaffolds. The inflammatory response, which influences the in vivo microenvironment and is associated with proinflammatory factors, such as TNF, is thought to reduce insulin secretion and delay maturation of the hPSC β-cells. Inflammation can attenuate glucose-stimulated insulin secretion of human β-cells, which has been reported to be inhibited by a combinatory treatment of tumor necrosis factor (TNF), lipopolysaccharide (LPS), and interferon (IFN). Pro-inflammatory chemokines and cytokines including IL-6, TNF-α and CXCL8 induce β-cell apoptosis by the activation of stress-induced kinases IKKβ and JNK, and initiation of a signaling cascade involving NFκB and NLRP3 in β-cells. Herein, media conditioned by the extracted scaffolds was used in the culture of stem cell derived β-cells to analyze the impact of the complex microenvironment rather than individual factors. The increase in apoptosis and decline in expression of maturation markers for the SA-engineered scaffold condition is consistent with the findings by others for β-cells and hPSC derived-cells. Moreover, SA-FasL-engineered scaffolds-maintained differentiation state and reduced caspase-dependent apoptosis. Proinflammatory cytokines, such as TNF-α, were reported to result in significant activation of caspase-dependent apoptosis via the extrinsic and intrinsic apoptosis signaling pathways. The caspase-8 levels found in the control, SA-FasL, and SA condition support the hypothesis that SA-FasL scaffolds reduced the expression of pro-inflammatory factors, which supported hPSC-derived β-cell survival. Overall, the decreased apoptosis demonstrated the potential of SA-FasL modified scaffolds as a platform for enhancing short-term graft survival post-transplantation.

SA-FasL-engineered scaffolds were observed to augment the engraftment and survival of β-cells by reducing inflammatory macrophages and monocytes, which can have upregulated Fas expression in response to inflammation. Specifically, the numbers of infiltrated macrophages and monocytes were reduced in the SA-FasL-engineered scaffolds relative to SA-engineered ones two weeks after transplant. FasL-mediated monocyte apoptosis was previously reported to be caspase dependent and associated with Fas-induced monocyte cytokine responses via nuclear translocation of NF-κB to attenuate early acute inflammatory responses and potential tissue injury. Furthermore, gene expression indicates that SA-FasL-engineered scaffolds promote the infiltrated macrophage polarization towards an anti-inflammatory (M2) phenotype and suppresses inflammatory pathways during the first week post-islet transplantation. The presence of M2 macrophages has been reported to associate with wound healing by exhibiting immune suppressive activity. In contrast, infiltrated neutrophil number was increased in the SA-FasL-engineered scaffolds compared to the SA-engineered control on day 4 and dropped rapidly for both conditions by day 14 post transplantation. FasL was reported to induce neutrophil recruitment to an inflammatory site via the activation of neutrophil mitogen-activated protein kinase for the potential inhibition of graft rejection. The observations of this study are consistent with other studies where neutrophils modulate monocyte and macrophage functions during the early phases of inflammation, while the interaction between macrophages and apoptotic cells will predominate in later stages. The previously reported FasL-induced patterns on monocytes and neutrophils support the immune cell infiltration trend found in this study. Early anti-inflammatory strategies for islet transplantation have proven critical to the success in some solid organ transplantations, and strategies to modulate inflammation may be particularly critical to facilitate the transplantation of stem cell derived β-cells.

SA-FasL-engineered scaffolds induced a sustained increase in insulin positive cells. Transplantation in vivo is necessary for insulin-producing hPSC-derived islets, and the local microenvironment at the transplant site affects the subsequent maturation of stem cell derived β-cells into functional islet clusters. Localized immune modulation has been proposed for augmenting the endogenous properties of the transplant microenvironment to further facilitate the maturation of hPSC-derived β-cells. In this study, differentiation of glucagon-producing o cells were curbed in the SA-FasL condition. The gene expression analysis captured the significance in glucagon expression levels, supporting the absolute cell number increase for a cells in the SA-scaffolds. The percentage of monohormal β-cells identified by immunostaining was increased with SA-FasL-engineered scaffolds s (FIG. 6A-B), suggesting the environment can influence the relative distribution of a and β-cells post transplantation. While the reduced inflammation by the SA-FasL may have increased cell survival by approximately 20%, other factors such as vascularization may have also decreased cell survival and differentiation post transplantation.

In conclusion, neutrophils, macrophages, and monocytes, that contribute to early dysfunction of β-cell transplants were influenced through the presentation of SA-FasL in the microenvironment. The number and the expression of-cell maturation markers (C-pep, NKX6.1) were increased on the SA-FasL-engineered scaffold compared to the control condition, with in vivo transplantation resulting in an increased cell number and greater numbers of monohormonal-cells. Stem cell derived β-cells are influenced by signals present within the microenvironment post-transplantation and modulating the immune response may be an important direction for maximizing survival and differentiation as a therapy for patients with Type I Diabetes.

### Example 6: Materials and Methods

### Biotinylation of PLG

The PLG polymer was biotinylated as previously described (Skoumal, M. et al. Biomaterials 192, 271-281 (2019)). Briefly, PLG (890 mg, 0.011 mmol) was added to a 20 mL glass scintillation vial and dissolved in 10 mL DMSO. The carboxyl end group of PLG was activated by first adding EDC (10.6 mg, 0.056 mmol) dissolved in 1 mL DMSO followed by NHS (6.4 mg, 0.056 mmol) dissolved in 1 mL DMSO, and the reaction was allowed to stir for 15 min. Amine-PEG2-Biotin (5 mg, 0.056 mmol) was dissolved in 1 mL DMSO and added dropwise to the stirring solution of PLG-NHS, and the reaction was allowed to stir overnight. Excess biotinylation reagent was removed by dialysis. The reaction mixture was poured into dialysis SnakeSkin tubing (7k MWCO) and let stir over night. The biotinylated PLG polymer from the dialysis tube was then lyophilized for 48 hours.

### Particle and Scaffold Fabrication

Biotin-PLG microparticles were formed for scaffold fabrication as previously described (Skoumal, M. et al. Biomaterials 192, 271-281 (2019)), Briefly, biotin-PLG conjugates were combined with unmodified PLG at a mass ratio of 3:1 (biotin-PLG: PLG) for a final concentration of 6 wt % in 2.04 mL of DCM (6 wt %) and sonicated in 10 mL of a 1% solution of PEMA at 100% amplitude (Cole-Parmer, β0 W, 3 mm stepped tip). The emulsion was poured into 200 ml of 0.5% PEMA and the organic solvent was evaporated by stirring the emulsion overnight. The particles were recovered by washing four times with deionized water by centrifugation at 7000×g for 15 min at 4° C. Particles were lyophilized for 48 hours and stored under vacuum.

Porous scaffolds were formed by mixing PLG particles with NaCl (250 μm<d<425 μm) at a 1:30 ratio (PLG:NaCl). The mixture was pressed in a 5 mm KBr die using a Carver press at 1500 psi for 30 seconds and foamed in CO₂ at 750 psi for 16 hours. Scaffolds were 5 mm in diameter and 2 mm in height with a pore size of 250-425 microns. Scaffolds were leached in water for 1 hour followed by a second wash for 30 minutes. Scaffolds were disinfected by soaking them in 70% ethanol and washed with PBS.

### Human Stem Cell Derived β-Cells

Pancreatic β-cells were differentiated from human pluripotent stem cells (hPSCs) using a 6-stage protocol from Dr. Jeffery Millman's lab (Hogrebe, N. J., et al. Nat. Biotechnol. 38, 460-470 (2020)).

The media used for each stage is as follows. Stage 1 (4 days): BEI+100 ng/mL Activin A+3 μM CHIR99021 for the first 24 hours, followed with 3 days of BEI containing 100 ng/mL Activin A only. Stage 2 (2 days): BE2+50 ng/mL KGF. Stage 3 (2 days): BE3+50 ng/mL KGF, 200 nM LDN193189, 500 nM TPPB (Tocris, 53431), 2 μM retinoic acid, and 0.25 μM SANT1. Stage 4 (4 days): BE3+50 ng/mL KGF, 200 nM LDN193189, 500 nM TPPB, 0.1 μM retinoic acid, and 0.25 μM SANT1. Stage 5 (7 days): S5+10 μM ALK5i II+20 ng/mL Betacellulin+0.1 μM retinoic acid+0.25 μM SANT1+1 μM T3+1 μM XXI. 1 μM Latrunculin A was added to this media for the first 24 hours only. Stage 6 (7-25 days): Cultures were kept on the plate with ESFM for the first 7 days. To move to suspension culture, cells could be single cell dispersed with TrypLE and placed in 6 mL ESFM within a 6-well plate at a concentration of 4-5 million cells/well on an orbital shaker at 100 RPM. Assessments were performed 5-8 days after cluster aggregation.

The base differentiation media formulations used above were as follows. BE1 medium: 500 mL MCDB 131 supplemented with 0.8 g glucose, 0.587 g sodium bicarbonate, 0.5 g BSA, and 5 mL GlutaMAX. BE2 medium: 500 mL MCDB 131 supplemented with 0.4 g glucose, 0.587 g sodium bicarbonate, 0.5 g BSA, 5 mL GlutaMAX, and 22 mg vitamin C. BE3 medium: 500 mL MCDB 131 supplemented with 0.22 g glucose, 0.877 g sodium bicarbonate, 10 g BSA, 2.5 mL ITS-X, 5 mL GlutaMAX, and 22 mg vitamin C. S5 medium: 500 mL MCDB 131 supplemented with 1.8 g glucose, 0.877 g sodium bicarbonate, 10 g BSA, 2.5 mL ITS-X, 5 mL GlutaMAX, 22 mg vitamin C, 5 mL P/S, and 5 mg heparin (MilliporeSigma, H3149). ESFM medium: 500 mL MCDB 131 supplemented with 0.23 g glucose, 10.5 g BSA, 5.2 mL GlutaMAX, 5.2 mL P/S, 5 mg heparin, 5.2 mL MEM nonessential amino acids (Corning, 20-025-CI), 84μg ZnSO4 (MilliporeSigma, 10883), 523 μL Trace Elements A (Coming, 25-021-CD), and 523 μL Trace Elements B (Corning, 25-022-CI).

### Mice and Recombinant Proteins

NSG mice (NOD Cg-Prkdc<scid>112rg<tm1Wj1>SzJ) were purchased from Jackson Laboratory and bred in a specific pathogen-free animal facility at the University of Michigan using protocols approved by the Institutional Animal Care and Use Committee. SA-FasL proteins were made with the Drosophila DES expression system (Invitrogen) following standard protocols (Yolcu, E.S., et al. Immunity 17, 795-808 (2002)).

### Scaffold Protein Loading and Cell Seeding

Biotin-PLG scaffolds were engineered by placing scaffolds described above to a well in a 12-well plate and adding SA-FasL (1 μg /scaffold) diluted in PBS and incubating at 20° C. for 30 minutes while rotating and shaking the plate every 10 minutes. The stem cell derived β-cells were then suspended at a density of 6 million cells per 30 μL media. The select FasL-loaded scaffold was next seeded side by side with about 2.5 million cells each side.

### Transplantation

NSG mice were chemically induced with diabetes by intraperitoneal (i.p.) injection of streptozotocin (140 mg/kg). Mice were monitored by reading blood glucose where ≥250 mg/dL for two consecutive days was considered diabetic. Diabetic mice were given anesthesia and a small incision was made on the abdomen to allow the cell-seeded scaffolds described above to be placed on epididymal fat pads (2 scaffolds/mouse). Adipose tissue was wrapped around scaffolds before being returned to the abdomen. Mice were then sutured and stapled.

### Media Conditioning for In-Vitro Experiments

To prepare conditioned media, explanted scaffolds were suspended in stage-6 media, and incubated at 37° C. for 24 h. After incubation, scaffolds were removed, and the conditioned media were stored at −80° C. Total protein concentration of the conditioned media was quantified by BCA protein assay kit (Thermo Fisher Scientific), and then diluted to 0.3 mg/ml for in vitro studies.

Stage 6 pancreatic β-cells were co-cultured with the conditioned media at a concentration of 500,000 cells with 1 ml of the conditioned media for 2 days before flow cytometry analysis and PCR analysis.

### PicoGreen Method for Cell Number Quantification

Cell containing scaffolds were mechanically homogenized in Trizol, and DNA was quantified based on the DNA standard curve according to the manufacturer's instructions using Quant-iT™ PicoGreen dsDNA Assay.

### Flow Cytometry Analysis

Scaffolds were extracted and harvested after indicated days. The embedded cells in the scaffold were processed into single cell suspensions using Liberase TL (Roche). ACK lysis buffer (ThermoFisher Scientific) was added to lyse red blood cells. The extracted cells were then stained with F480, Ly6C, Ly6G, CD11b and analyzed by ZE5 cell analyzer (Bio-Rad).

### Caspase-8 Measurement

Caspase-8 level was measured in β-cells per the manufacturer's instruction (Abcam). Briefly, β-cells were extracted, homogenized and lysed before assay development for plate reading at 405 nm.

qRT-PCR Analysis

Gene expression analysis was performed as described previously (Youngblood, R. L., et al. Acta Biomater, 96, 111-122 (2019)). Briefly, cell containing scaffolds were mechanically homogenized, and RNA was isolated according to the manufacturer's instructions. RNA concentration was standardized by a NanoDrop spectrophotometer. The Applied Biosystems High Capacity kit was used to transcribe RNA into cDNA. Gene expression was quantified using the AACt method and fold change was calculated using the formula 2-ΔΔCt. Values for the genes of interest were normalized to the housekeeping gene (GAPDH) followed by normalization to marker expression in pluripotent hPSCs.

### Statistical Analysis

Statistical significance was determined using two-sided unpaired t-tests. A p-value that is less than 0.05 was considered statistically significant. Results were presented as the mean±SEM.

### Immunostaining

Scaffold extracted were cryopreserved in isopentane cooled on dry ice and then embedded within OCT embedding medium and cryosectioned. Digital images were acquired with a MicroFire digital camera (Optronics, Goleta, CA) connected to an Olympus BX-41 fluorescence microscope (Olympus, Center Valley, PA, United States). Image quantification was conducted using MATLAB software using an object-based colocalization analysis, DAPI+ cells were identified out of the total area of the sectioned tissue and quantified by applying Otsu's thresholding method, the watershed transforms, and individual cluster thresholding. Then, each cell's colocalization with immunofluorescent markers was quantified.

What is claimed is:

1. A composition comprising a FasL-loaded poly(lactide-co-glycolide) (PLG) scaffold and immature β-cells.

2. The composition of claim 1, wherein the immature β-cells are hPSC-derived cells.

3. The composition of claim 1 or 2, wherein the immature β-cells are human induced pluripotent stem cell (iPSC)-derived cells or cells producing insulin.

4. The composition of any one of claims 1 to 3, wherein the immature β-cells comprise at least six million immature β-cells.

5. The composition of claim 4, wherein the immature β-cells comprise from about 0.1 million to about 10 million immature β-cells per scaffold.

6. The composition of any one of claims 1 to 5, wherein the composition comprises about 400 million to about 800 million immature β-cells.

7. The composition of any one of claims 1 to 6, wherein the FasL-loaded PLG scaffold is about 5 mm in diameter.

8. The composition of any one of claims 1 to 6, wherein the FasL-loaded PLG scaffold is between about 10 mm and about 35 mm in diameter.

9. The composition of any one of claims 1 to 8, wherein the FasL-loaded PLG scaffold is about 2 mm in height.

10. The composition of any one of claims 1 to 9, wherein the FasL-loaded PLG scaffold has a pore size of between about 250 microns and about 425 microns.

11. The composition of any one of claims 1 to 10, wherein the FasL-loaded PLG scaffold comprises about 1 μg to about 50μg of FasL.

12. The composition of any one of claims 1 to 11, wherein the composition further comprises more than one FasL-loaded PLG scaffold.

13. The composition of claim 12, wherein the composition comprises from about 20 scaffolds to about 2000 scaffolds.

14. A method of making a composition comprising a FasL-loaded poly(lactide-co-glycolide) (PLG) scaffold and immature β-cells, wherein the method comprises:
a) loading poly(lactide-co-glycolide) (PLG) with biotin;
b) making a biotin-loaded PLG scaffold from the biotin-loaded PLG;
c) conjugating streptavidin-FasL to the biotin on the biotin-loaded PLG scaffold to form a FasL-loaded PLG scaffold; and
d) seeding the FasL-loaded PLG scaffold with immature β-cells.

15. The method of claim 14, wherein loading the PLG with biotin comprises activating the carboxyl end group of PLG with EDC followed by NHS and then adding amine-PEG2-biotin.

16. The method of claim 14 or 15, wherein making the biotin-loaded scaffold from the biotin-loaded PLG comprises:
a) combining the biotin-loaded PLG with unmodified PLG at a mass ratio of 3:1 and forming biotin-loaded PLG particles,
b) mixing the biotin-loaded PLG particles with NaCl at a mass ratio of 1:30 and pressing the mixture into a 5 mm KBr die to form the biotin-loaded scaffold; and
c) disinfecting the biotin-loaded scaffold by soaking it in 70% ethanol.

17. The method of any one of claims 14 to 16, wherein conjugating streptavidin-FasL to the biotin on the biotin-loaded PLG scaffold comprises adding about 1 μg to about 50μg streptavidin-FasL diluted in PBS to the biotin-loaded PLG scaffold and incubating it at 20° C.for 30 minutes while rotating and shaking the plate every 10 minutes.

18. The method of any one of claims 14 to 17, wherein seeding the FasL-loaded PLG scaffold with immature β-cells comprises adding immature β-cells at a density of from about 1 million to about 10 million cells per 30 μL of media and seeding from about 0.5 million to about 5 million cells per side of the FasL-loaded PLG scaffold.

19. The method of any one of claims 14 to 18, wherein the immature β-cells are hPSC-derived cells.

20. The method of any one of claims 14 to 19, wherein the immature β-cells are human iPSC-derived cells or cells producing insulin.

21. A method of treating a subject with type I diabetes, wherein the method comprises implanting a composition comprising a FasL-loaded PLG scaffold of any one of claims 1 to 13 into the subject.

22. The method of claim 21, wherein the scaffold is transplanted into the subject's peritoneal fat.

23. The method of claim 21 or 22, wherein the scaffold is transplanted into white adipose tissue of the subject's omentum or epididymal fat pad.

24. The method of any one of claims 21 to 23, wherein the subject is a mammal.

25. The method of any one of claims 21 to 24, wherein the subject is a human.

26. The method of any one of claims 21 to 25, wherein the immature β-cells are derived from the subject via iPSCs.
